(19) United States
(12) Patent Application Publication
(10) Pub. No.: US 2021/0386541 A1
(43) Pub. Date: Dec. 16, 2021

(54) IMPLANT FOR THE TREATMENT AND/OR FOR REPLACEMENT OF AN INFLAMMED, THROMBOSED OR DEGENERATED HEART VALVE

(71) Applicant: DEVIE MEDICAL GMBH, Jena (DE)

(72) Inventors: Hans Reiner Figulla, Jena (DE); Raphael A. Seidel, Jena (DE)

(21) Appl. No.: 17/283,947

(22) PCT Filed: Apr. 5, 2019
(86) PCT No.: PCT/EP2019/058666

§ 371 Date: Apr. 8, 2021

Related U.S. Application Data
(60) Provisional application No. 62/743,828, filed on Oct. 10, 2018.

(51) Int. Cl. A61F 2/24; A61F 2/00

(52) U.S. Cl. CPC A61F 2/2418; A61F 2/2409; A61F 2/2427; A61F 2220/0016; A61F 2002/0091; A61F 2210/0004; A61F 2250/0067; A61F 2/0077

(57) ABSTRACT

The invention relates to an implant (1) for treating and/or for replacing a heart valve (100) diseased by an inflammation and/or infection, wherein the implant (1) has a catching device (2) which, in a compressed state, can be introduced in a minimally invasive manner into the body of the patient, and which can be expanded at the implantation site on the diseased heart valve (100), wherein the catching device (2) is designed, at least in the implanted and expanded state, to compartmentalise tissue changes or tissue deposits (101), in particular in the form of heart valve vegetation or deposits, in particular by using the catching device (2) to engage around and/or receive at least regions of the tissue change or deposit (101).

The present invention stems from the field of cardiosurgery and cardiology and relates in particular to the treatment and/or replacement of a heart valve diseased by inflammation and/or infection.

There are four valves in the native heart which serve to direct blood flow through the two sides of the heart in a forward direction. The left (systemic) side of the heart has the mitral valve, which lies between the left atrium and the left ventricle, and the aortic valve, which lies between the left ventricle and the aorta. These two valves direct oxygen-rich blood coming from the lungs through the left side of the heart and into the aorta for distribution to the body. The right (pulmonary) side of the heart has the tricuspid valve, which lies between the right atrium and the right ventricle, and the pulmonary valve, which lies between the right ventricle and the pulmonary artery. These two valves direct oxygen-deficient blood coming from the body through the right side of the heart and into the pulmonary artery for distribution to the lungs where it is re-oxygenated to start the circulation anew.

All four of these heart valves are passive structures in that they themselves do not expend any energy nor perform any active contractile function. They consist of movable “cusps,” at times also referred to as “leaflets,” and are meant to easily open and close in response to different pressures on either side of the valve. The mitral and tricuspid valves are referred to as “atrial ventricular valves” due to their location between an atrium and a chamber on either side of the heart. The mitral valve has two leaflets and the tricuspid valve has three leaflets. The aortic and pulmonary valves are described as “crescent-shaped valves” and are appropriately called “leaflets.” The aortic and pulmonary valves each have three leaflets.

Endocarditis is an inflammation of the heart's innermost layer (endocardium) which lines the chambers of the heart and those arteries and veins close to the heart and also forms the structure of the heart valve leaflets. Numerous micro-organisms can cause endocarditis—particularly gram-positive bacterial species such as streptococci, enterococci and staphylococci. Should these colonize the endocardium in the course of bacteremia, infective endocarditis develops.

Depending on the type of elicitor, a life-threatening consequence occurs in approximately 25% of the cases.

According to the prior art, one possibility for treating endocarditis is the surgical removal of the inflamed heart valve and the implantation of an artificial heart valve or replacement heart valve respectively. As a rule, this entails labor and cost-intensive surgeries which are coupled with high patient stress and a considerable risk. In detail, the patient's chest is opened, the heart is stopped with a cardioplegic solution, the endogenous heart valve removed and an artificial heart valve sewn to the body's own tissue in its place. Newer methods such as presented in WO2006/076890 A1 provide for a transcatheter implantation of artificial heart valves using a stent as a supporting structure, although this can only be used in the case of degenerated aortic valve diseases.

In the case of inflamed heart valves with bacterial attachments or deposits in the sense of larger vegetations, the only applicable surgical procedures to date have been those which remove the infected, inflamed material in the surrounding tissue of the heart valve as well as the heart valve itself and then sew in an artificial heart valve. Catheter-guided valve implantations are contraindicated with inflamed heart valves since, as noted above, the inflamed material cannot thereby be removed. Especially with endocarditis, however, the surgical procedures used in 75% of endocarditis cases are particularly risky (25% do not undergo operation because the patients are either no longer operable or because there is only mild inflammation). This is due to endocarditis patients usually having significant comorbidities associated with the heart valve inflammation such as embolizations in the brain, kidneys, skin and other organs and because the patients are septic; i.e. have a high fever and inflammation-related impairments, a destroyed valve which has led to heart failure requires emergency surgery.

On the basis of this problem at set forth, the invention is thus based on the task of implanting a catheter-guided heart valve which can locally treat the infection and/or thrombosis and the degenerated heart valve at the same time.

With regard to the implant, the task on which the invention is based is solved in particular by the subject matter of independent claim 1, whereby advantageous developments of the inventive implant are specified in the respective subclaims.

With regard to the method, the task on which the invention is based is solved in particular by the subject matter of accompanying independent claim 14.

Accordingly, the invention relates in particular to an implant for treating or replacing an inflamed and/or infected heart valve, wherein the implant comprises a capture device able to be introduced into the patient's body in a compressed state in minimally invasive manner and expanded at the implantation site on the diseased heart valve. The capture device, at least in the implanted and expanded state, is in particular designed to compartmentalize tissue deposit particularly in the form of heart valve vegetation, and that in particular by the capture device at least partially grasping and/or capturing the tissue deposit.

Various embodiments are possible for the capture device of the inventive implant. It is for example conceivable for the capture device to exhibit a reticulated or latticed or membranous structure by means of which the tissue deposit on the diseased native heart valve can be at least partially grasped and/or captured in order to compartmentalize it accordingly.

Alternatively or additionally thereto, it is however also conceivable for at least part of the capture device to exhibit a fibrous structure, particularly of a bioresorbable material, in order to enable an encapsulating of the tissue deposit in the implanted and expanded state of the implant or capture device respectively in the region of the native heart valve to be treated. The fibrous structure of the capture device can for example be composed of bioresorbable fibers, in particular microfibers, which are for example cross-linked so as to form a non-woven or mat-like structure.

Other embodiments as well as combinations of same are, however, of course also possible for the capture device.

In order to enable the most precise and secure fixation and/or positioning of the capture device in the implantation site, the capture device is preferentially allocated a suitable anchoring structure. It can thereby be provided for a capturing structure to be suitably connected to the anchoring structure so as to jointly form the capture device. According to embodiments of the capture device, when the implant is in implanted state, the capturing structure connected to the anchoring structure should safely extend downstream, as viewed in the direction of the blood flow, into a vessel connected to the diseased heart valve in terms of flow.

Moreover, in the expanded state, the capturing structure can exhibit at least one clamping area designed to interact with at least one heart valve leaflet of the diseased heart valve in the capture device's expanded and implanted state such that the at least one heart valve leaflet is pressed toward a vascular wall. The clamping area of the capture device is preferably designed such that ideally all the heart valve leaflets of the diseased heart valve are suitably pressed toward the vascular wall. In so doing, not only can the tissue deposit (in particular heart valve vegetation) on the leaflets of the diseased heart valve be compart-mentalized but the diseased heart valve is at the same time also prepped for replacement with a replacement heart valve (prosthetic heart valve).

The clamping area over which the heart valve leaflet(s) of the diseased native heart valve is/are pressed toward the vascular wall can be designed in different ways. It is particularly advisable for the at least one clamping area to comprise at least one clamping arm or clamping bracket designed to at least partly expand radially upon the capture device being expanded in the implantation site.

In order to be able to compartmentalize the tissue deposit as effectively as possible, embodiments of the invention provide for the clamping area to exhibit at least one area at a distal end region on the far side from the anchoring structure which is directed at least substantially toward said anchoring structure.

According to embodiments of the inventive implant, same further has a structure able to be in particular implanted separately from the capture device which in particular exhibits a substantially annular structure and is insertable into the pockets of the native heart valve (between the leaflets and the vascular wall) and serves as abutment particularly for the capture device and/or the clamping area of the implant.

For example, this structure particularly able to be implanted separately from the capture device can be realized as an at least substantially annular abutment implant which can be implanted in the implantation site such that in the implanted state of the implant, at least some areas of at least one valve leaflet of the heart valve to be treated can be accommodated between the implant and the abutment implant.

According to a further aspect of the present invention, the implant serves in particular to replace an inflamed, thrombosed or degenerated heart valve, and in particular an endocarditis-diseased heart valve, wherein in addition to the above-discussed capture device, the implant comprises an expandable replacement heart valve, or prosthetic heart valve respectively, which can be introduced into the patient's body in a compressed state in a minimally invasive manner and expanded at the implantation site on the diseased heart valve such that the replacement heart valve, or prosthetic heart valve respectively, at least substantially assumes the function of a native heart valve.

According to embodiments, the replacement heart valve is allocated at least one suitable stent in order to appropriately support and hold the replacement heart valve particularly in the implanted state. The stent thus constitutes the carrier and supporting structure of the replacement heart valve and at the same time serves to position and anchor the replacement heart valve at the implantation site.

However, it is of course also conceivable in this context for the replacement heart valve to not only be allocated just a single stent but rather multiple, in particular two or three, stents so as to be able to realize a so-called stent-in-stent solution in the implantation site, wherein a first stent is fixed as an abutment in the area between the aortic wall and the native heart valve leaflet and a second stent with the replacement heart valve secured thereto being inserted into the already implanted stent. Such a stent-in-stent solution offers the advantage of the replacement heart valve being firmly seated and biological material being able to be more efficiently compartmentalized. A further advantage can be seen in the implant being able to be implanted component by component in order to replace an endocarditis-diseased heart valve so as to enable the use of a catheter introduction system having a maximum catheter diameter of in particular less than 22 Fr, since both components can also be carriers of active substances and thus an overall larger volume can be introduced with the same catheter diameter.

According to implementations of the inventive implant, it is provided for same to comprise a suitable replacement heart valve with a respectively allocated stent in addition to the capture device for compartmentalizing the tissue deposits, wherein this stent constitutes the carrier and supporting structure of the replacement heart valve. Moreover, the stent allocated to the replacement heart valve can be designed to displace the heart valve leaflets of the native heart valve in the implantation site, particularly in the radial direction and preferably in such a way that the heart valve leaflets of the native heart valve are pressed against or toward the vascular wall.

Also appropriate in this context is for the stent allocated to the replacement heart valve to simultaneously form an or the anchoring structure of the capture device. In other words, the stent allocated to the replacement heart valve can have a dual function: on the one hand, the stent can serve as a carrier and supporting structure of the replacement heart valve, wherein the positioning and anchoring of the replacement heart valve in the implantation site on the diseased native heart valve is ensured simultaneously. On the other hand, the stent allocated to the replacement heart valve can serve as an anchoring structure for the capture device, wherein a corresponding capturing structure is then attached or attachable to the stent serving as the anchoring structure of the capture device.

In order to achieve the implant being introduced into the patient's body as minimally invasively as possible using the thinnest possible catheter introduction system, it is expedient for the implant to be designed to expand incrementally over time in the implantation site on the diseased native heart valve. Embodiments thereby provide for the capture device to expand in a first step in order to capture and compartmentalize the tissue deposits, whereby an expansion of the replacement heart valve then follows. However, the invention is of course not limited to this sequence. In fact, the implant can also be designed such that the replacement heart valve expands in a first step and thereby pushes the heart valve leaflets of the native heart valve with the tissue deposits radially outwardly, wherein the radially outward-pressed heart valve leaflets, and in particular tissue deposits on the heart valve leaflets of the native heart valve, are then appropriately compartmentalized via the capture device—for example also in conjunction with an abutment structure previously inserted separately.

In one preferential development, the inventive implant is further designed to release active substances in the implanted state. This allows the inflammation, thrombosis or degeneration of the native heart valve to be treated locally; i.e. in situ and topically, and the infection, thrombosis or degeneration to be controlled without necessitating additional stress/risk to the patient from surgical treatment of the native heart valve.

Conceivable active substances in particular include antimicrobial, antibiotic, bactericidal, antithrombotic, thrombolytic, cyotoxic and/or comparable active substances which are able to be released to the surrounding tissue and bloodstream at the site of the disorder with the aid of the inventive implant.

For example, it is conceivable in this context for a coating of active substances to provided on the capture device and/or a replacement heart valve that may be part of the implant or a stent allocated to such a replacement heart valve respectively for the purpose of releasing the active substances.

According to embodiments of the present invention, a bioresorbable structure which is part of the inventive implant and which in particular comprises a fibrous and/or non-woven structure and/or membrane structure having an active substance is used to release the active substances.

The inventive implant is in particular designed to release the above-cited active substances to the surrounding tissue and into the bloodstream at the site of the disorder.

To that end, the inventive implant can comprise a stent/active substance system having at least one stent to which the capture device and/or a replacement heart valve is/are secured. The inventive implant in particular comprises biocompatible materials in order to ensure good post-implantation integration of the system into the biological environment. The at least one stent constitutes the carrier and supporting structure of the replacement heart valve and simultaneously serves in the positioning and anchoring of the inventive system, and in particular the capture device, at the implantation site.

The stent/active substance system must be able to radially displace the insufficient native heart valve for the capture device to compartmentalize the heart vegetation and clamp the replacement heart valve instead of the native heart valve and to ensure unfailing valve function during the systole and diastole of the heart. The at least one stent also needs to be suited to providing secure retention for the replacement heart valve during the periodic beating of the heart so that the inventive system cannot dislodge from the biological tissue, in particular from the vascular wall, and be flushed out of the implantation site due to changing pressure conditions in the heart.

To that end, the inventive system can comprise at least one stent able to be expanded by balloon expansion using a balloon catheter and positioned at the implantation site. The stent, which is compressed and encapsulated in the catheter, is thereby expanded by a catheter balloon to be filled with liquid or gas.

Alternatively, the at least one stent of the inventive system can be a self-expanding stent. The stent in particular consists of a shape memory alloy, preferably nitinol, to that end. In addition to the shape memory effect at a specific transition temperature, which is close to body temperature, nitinol also exhibits superelasticity, biocompatibility and corrosion resistance. Nitinol is thus already widely used in medical technology. Particularly the superelasticity is advantageous with respect to a stent's compressed form of delivery in the transcatheter method and the expansion at the implantation site. In addition to the two separately executed expansion processes, a combination of both processes is likewise possible. In particular, the stent's radial pretension can be additionally further increased after the self-expansion by a balloon expansion, whereby a higher stability of the inventive system in the implanted state is in turn achieved.

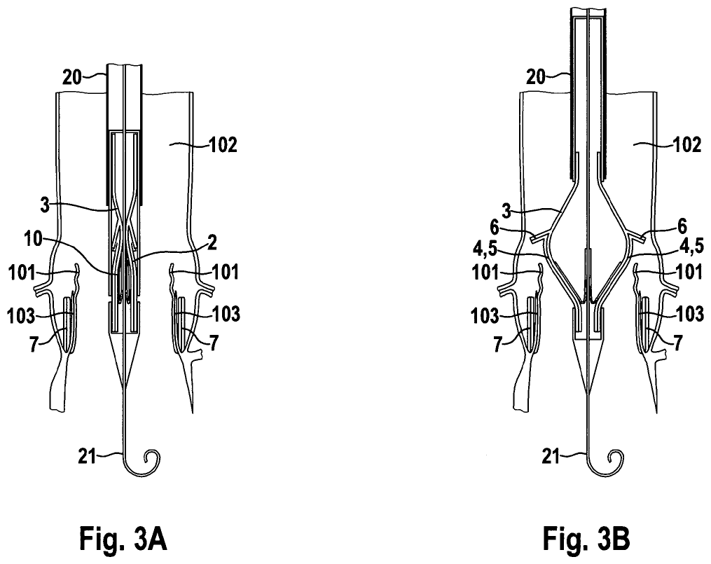

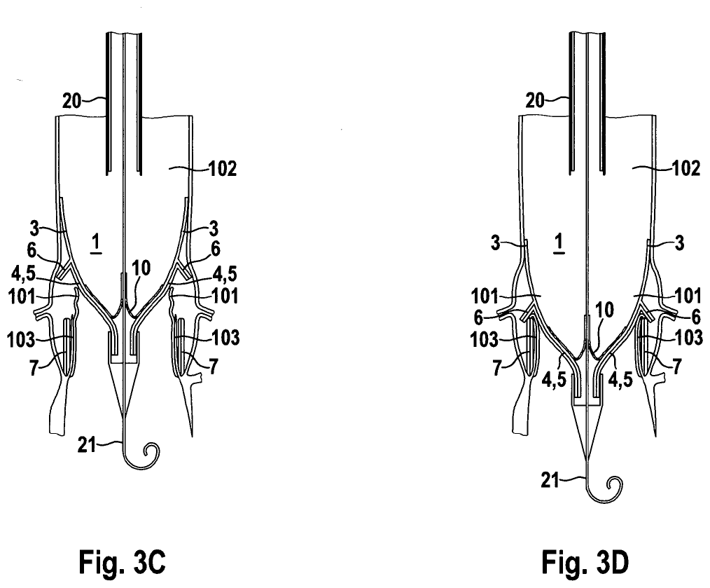

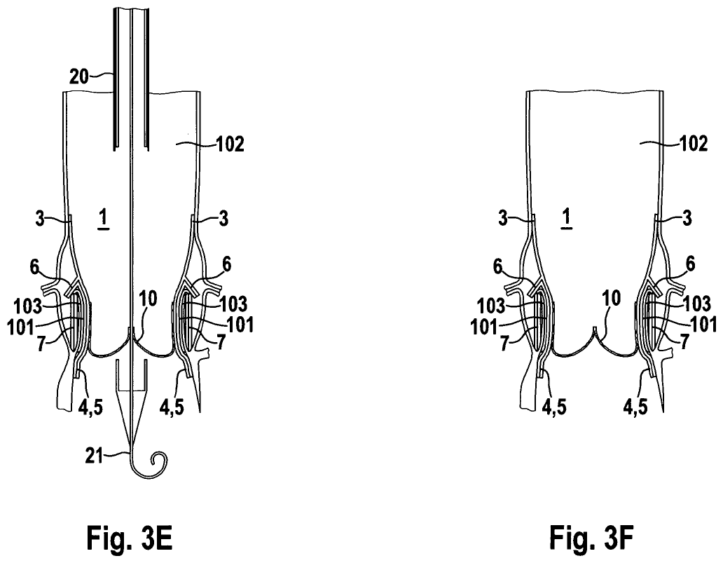

In conjunction hereto, it is also possible for a fibrous structure made of nitinol, another metalliferous substance or stable polymer struts to serve as scaffolding for supporting the replacement heart valve and capture device instead of a strut-like stent structure with a capture device. As depicted in FIGS. 3A to 3F, a tissue-like stent structure is particularly suited to achieving a reducing of the capture support to the annular structure of the capture device.

The replacement heart valve secured to the at least one stent can be a pericardial valve, a biological heart valve, an artificial heart valve, preferably of biocompatible materials, or a comparable implant or transplant suitable for replacing an insufficient heart valve. The system thus offers the advantage of the replacement heart valve being able to have the optimal design depending on the patient-specific conditions. Furthermore, the replacement heart valve has at least two leaflets. With regard to the replacement of a three-part heart valve, usage with more than two, in particular three, leaflets is also conceivable. The use of the inventive system is thus not limited just to the replacement of one insufficient native aortic valve, particularly not by the number of leaflets.

In their intended use, the leaflets of the replacement heart valve have two positions in particular, which they assume during the systole and diastole of the heart. With the objective of mimicking a native heart valve as a biological model, an equivalent conferring of leaflet functionality as per the biological model is accordingly also conceivable for the replacement of the other native heart valves. In a first position of the leaflets, during the diastole of the heart, the flow connection between the left ventricle and the aorta is completely closed so as to prevent blood reflux. The commissures of the leaflets, the inner vascular edges, are thereby in contact with one another. During the systole of the heart, the leaflets assume a second, opened position so that the blood can be pumped from the ventricle into the aorta. The commissures of the leaflets have no contact with one another in this second position.

One embodiment of the at least one stent preferably exhibits an interior and/or exterior coating consisting of an antimicrobial substance or an antimicrobial substrate. The at least one stent can thus release one or more antimicrobial active substances and achieve improved integrability of the implanted system in contact with the surrounding vascular wall. Possible particularly when using an antimicrobial substrate is a combination encompassing further components seeded at and/or onto the substrate such as anticoagulants, other antimicrobial substances such as bactericides, etc. Methods applicable to the inventive system in order to be able to produce such coatings include applying a film/membrane to the stent surface as well as other physical and/or chemical deposition procedures for applying a surface coating. It is thus possible to achieve complex release kinetics even of different active substances on the stent surface.

In a further embodiment, a stent surface coating can be activated in a controlled manner. A preferably antimicrobial effect then only occurs upon the surface coating being activated. Such a controlled activation of the surface coating can preferably ensue by ultrasound being administered from outside of the patient's body, whereby at least one toxic substance bound to specific carrier media on the stent surface, such as carbon dioxide, is released.

Application can also ensue in the form of microfibers or non-woven material from which the active substance can be released, whereby this can in particular be bioresorbable polymer materials into which the active substance(s) were introduced such as polylactides, polyglycolides, polylactones, polydioxanones, polyglycols, combinations and/or chemical modifications thereof.

Alternatively or additionally thereto, the inventive system has a first skirt region inside and a second skirt region outside of the provided stent, wherein at least one applied or introduced or respectively filled substance is released into the ventricle-side retention area of the stent. In the case of an aortic valve replacement, the ventricular retention area is particularly to be understood as the retention area of a stent or a stent system according to the invention facing the left ventricle of the heart and the opposite aortic retention area. This thus results in blood flow through the inventive system, whereby at least one substance is released at the ventricle-side blood inlet of the system while the flow of blood on the blood outlet side of the inventive system passes into the aorta at the aorta-side retention area. Pursuant to the inventive use of the claimed system, the at least one substance is always to be released on the blood inlet side of the inventive system even when other heart valves are replaced.

The invention further relates to a suitable catheter for introducing an implant into the body of a patient, particularly an implant of the aforementioned type, which serves to treat or to replace an inflamed, thrombosed or degenerated heart valve. The catheter has a catheter tip to that end which can be manipulated via a handle of the catheter such that the implant can be released from the catheter tip sequentially; i.e. incrementally.

According to a further aspect of the present invention, same relates to a corresponding system for treating or replacing an inflamed and/or infected heart valve, wherein this system comprises an implant according to the present invention as well as a catheter of the aforementioned type. The implant is thereby preferably designed so as to be able to be accommodated in the catheter tip of the introducer catheter when extended in the implant's longitudinal direction and in compressed or reduced state relative to the implant's radial direction. The catheter tip of the introducer catheter is in turn preferably designed to accommodate the implant when extended in the implant's longitudinal direction and in reduced state relative to the implant's radial direction.

Since the implant can be accommodated in the catheter tip in an elongated state according to these embodiments, the effective outer diameter of the catheter tip and in particular also of a catheter system which connects the catheter tip to the handle of the catheter can have a particularly small diameter. According to preferential implementations, the diameter of the catheter is 18F or less.

In the method according to the invention for treating an inflamed and/or infected heart valve, it is in particular provided for hyperplastic tissue changes, particularly in the form of heart valve vegetation, to first be compartmentalized in the diseased (native) heart valve, and this in particular by the hyperplastic tissue changes being at least partially captured and/or grasped by a suitable capture device to be implanted in the patient's body.

A replacement heart valve can thereafter be implanted to replace the function of the diseased native heart valve. The method can furthermore have the further method step of releasing active antimicrobial substances, wherein this is preferably an in situ release.

The inventive implant is in particular designed to expand incrementally over time in the implantation site on the diseased native heart valve, wherein the capture device expands in a first step and the replacement heart valve expands in a second step.

Alternatively thereto, the implant is designed to expand incrementally over time in the implantation site on the diseased native heart valve, wherein the replacement heart valve expands in a first step and the capture device expands in a second step.

According to preferential implementations, the implant has a structure able to be in particular implanted separately from the capture device and heart valve which in particular exhibits a substantially annular self-expanding structure and is insertable into the pockets of the native heart valve and serves as an abutment particularly for the capture device or the clamping area of the implant.

The invention further relates to a catheter for introducing an implant into the body of a patient, in particular an implant for treating or replacing an inflamed, thrombosed or degenerated heart valve of the aforementioned type, wherein the catheter has a catheter tip which can be manipulated via a handle of the catheter such that the implant can be released from the catheter tip incrementally.

The invention moreover relates to a system for treating or replacing an inflamed, thrombosed or degenerated heart valve with an implant of the inventive type and a catheter of the aforementioned type, wherein the implant is designed in particular to be able to be accommodated in the catheter tip in a state extended in the implant's longitudinal direction and reduced relative to the implant's radial direction, and wherein the catheter tip is designed to accommodate the implant particularly in its state extended in the implant's longitudinal direction and reduced relative to the implant's radial direction.

According to embodiments, the system comprises an additional and/or independent, in particular at least substantially annular, self-expanding implant, which is also referred to herein as “abutment implant,” and is designed to serve in particular as an abutment for the implant and which is in particular able to be folded by radial stretching and accommodated in folded form in a catheter and implanted at the implantation site such that in the implanted state of the implant, at least some areas of at least one valve leaflet of the heart valve to be treated can be accommodated between the implant and the additional and/or independent implant.

The abutment implant can be designed to be receivable in the catheter tip in the compressed state so that the abutment implant in the compressed state is not aligned coaxially but rather orthogonally to the catheter tip axis and is also able to be incrementally released at the implantation site by means of one or more pressure-resistant connections.

The invention further relates to a method for treating or replacing an inflamed, thrombosed or degenerated heart valve, wherein the method comprises the method step of compartmentalizing tissue changes or tissue deposits, particularly in the form of heart valve vegetation or deposits, and this in particular by the tissue changes or deposits being at least partially captured and/or grasped by a capture device.

The method preferably also comprises the method step of implanting a replacement heart valve to replace the diseased native heart valve, wherein the step of compartmentalizing and the step of implanting the replacement heart valve and abutment are preferably performed chronologically sequentially.

The method in particular further comprises the method step of releasing antimicrobial, antithrombotic and cell growth-inhibiting active substances, wherein the release ensues in situ.

The following will reference the accompanying drawings in describing exemplary embodiments of the invention in greater detail.

Shown are:

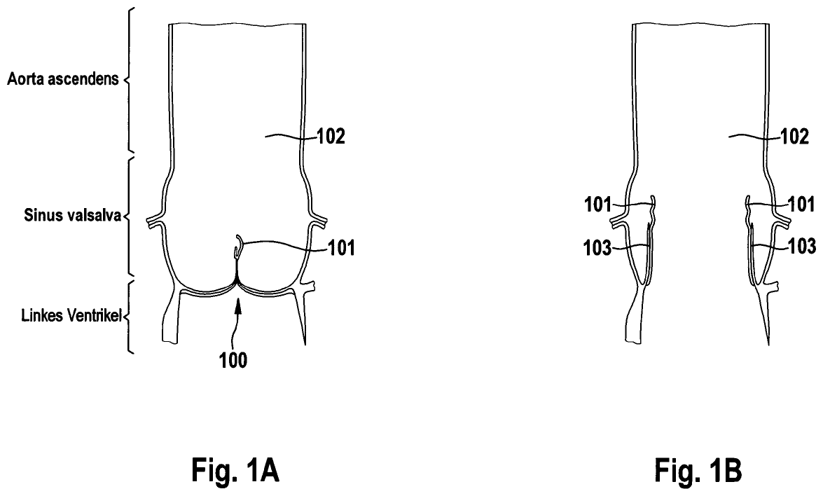

FIGS. 1A and 1B a schematic and sectional view of an endocarditis-diseased aortic valve with bacterial vegetation, wherein FIG. 1A shows the aortic valve in the closed state and FIG. 1B shows the aortic valve in the open state;

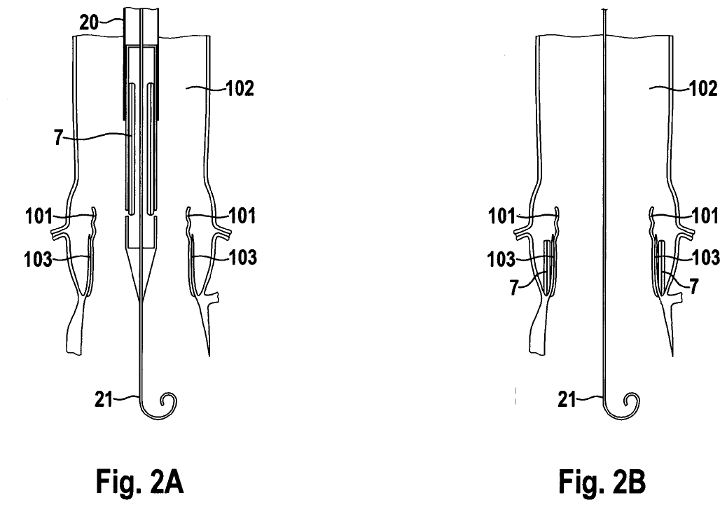

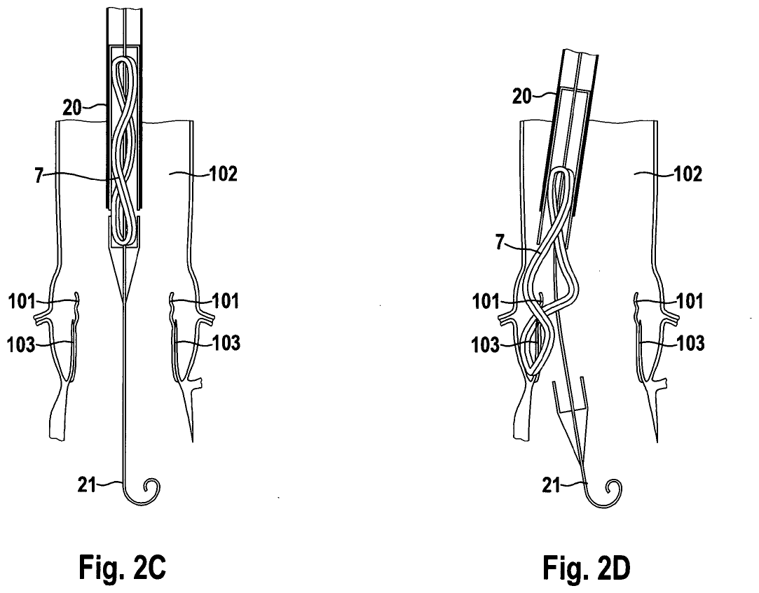

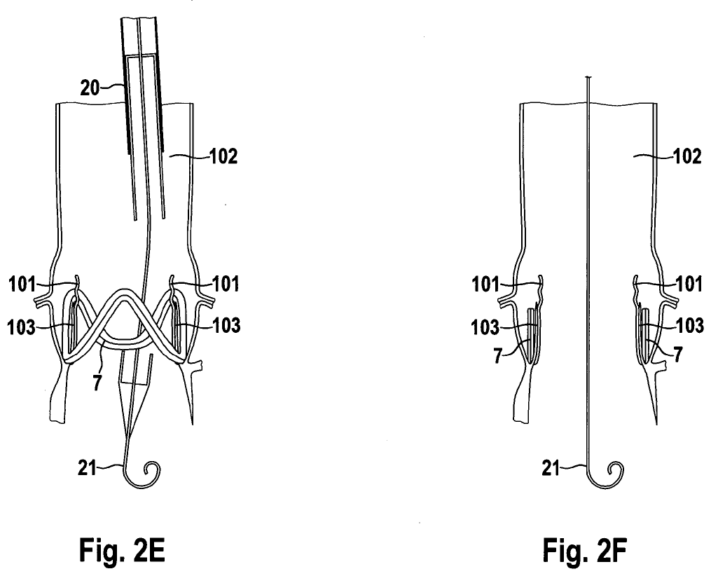

FIGS. 2A and 2B a schematic and sectional view of the inserting (FIG. 2A) and final position (FIG. 2B) of an active substance-coated implant serving as an abutment for a further stent;

FIG. 2C to 2F a schematic and partially sectioned view (FIG. 2C to 2E) or respectively sectioned view (FIG. 2F) of an active substance-coated implant serving as an abutment for a further stent during introduction orthogonal to the catheter axis, pushing out of the catheter, and placement behind the aortic valve leaflets;

FIG. 3A to 3F a schematic and sectional view of the insertion, placement and release of a first exemplary embodiment for the treatment and replacement of an endocarditis-diseased aortic valve;

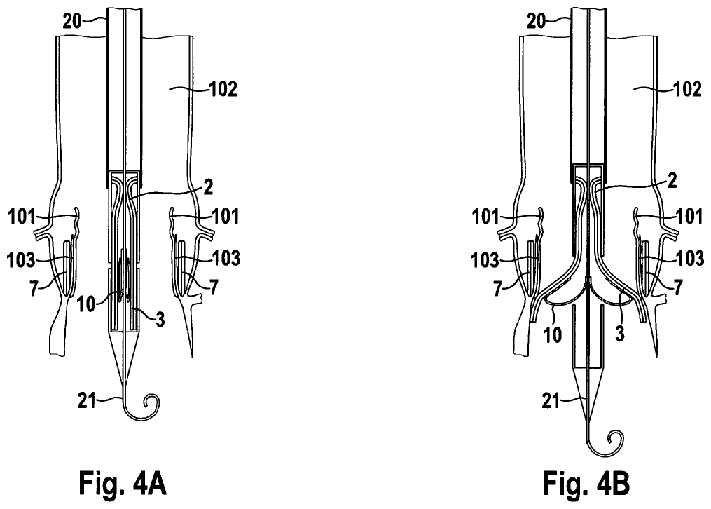

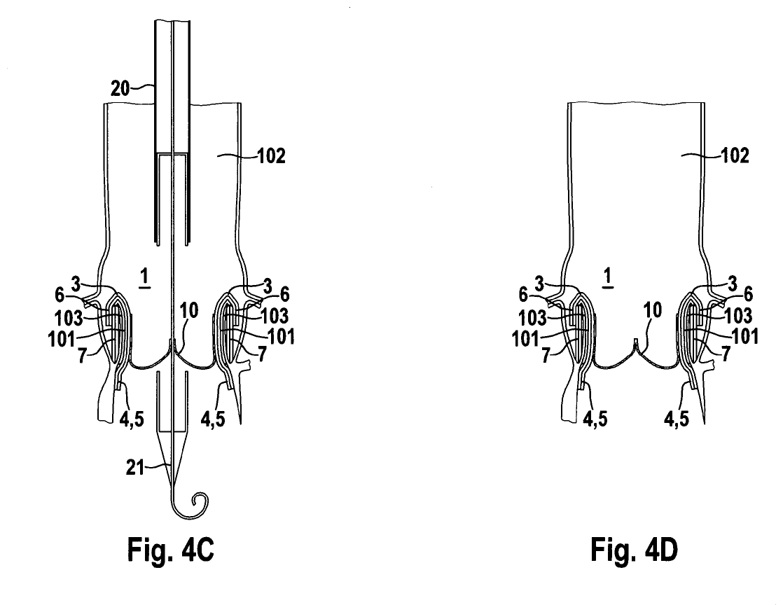

FIG. 4A to 4D a schematic and sectional view of the insertion, placement and release of a second exemplary embodiment for the treatment and replacement of an endocarditis-diseased aortic valve; and

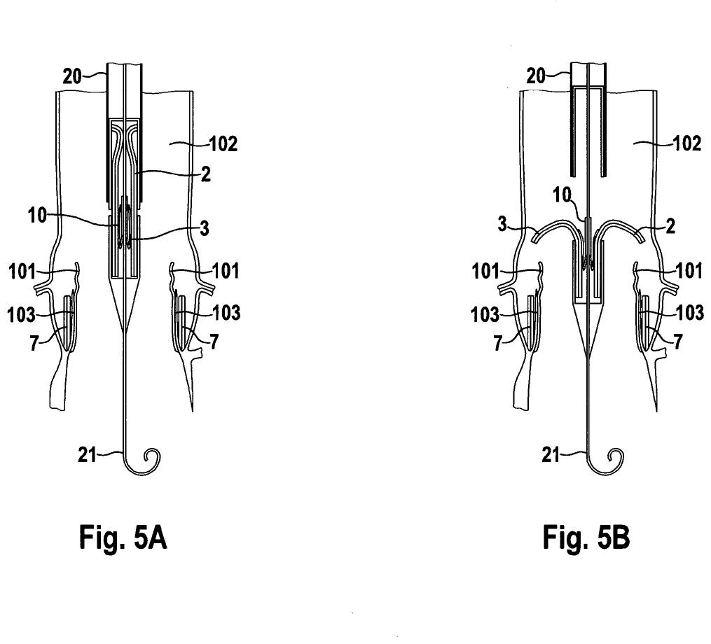

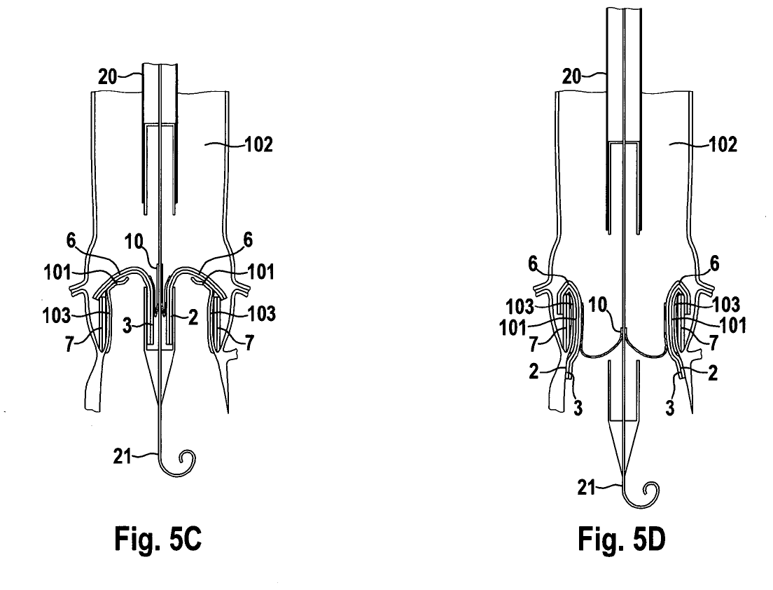

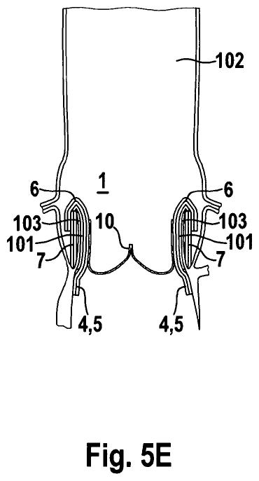

FIG. 5A to 5E a schematic and sectional view of the insertion, placement and release of a third exemplary embodiment for the treatment and replacement of an endocarditis-diseased aortic valve.

FIG. 1 shows the anatomy of an endocarditis-diseased aortic valve 100 in schematic and sectional view, wherein the aortic valve is depicted closed in FIG. 1A and the aortic valve is depicted open in FIG. 1B.

Briefly summarized, the aortic valve 100 is one of the four valves of the heart. Located in the aorta 102 directly at its root from the left ventricle, it prevents the reflux of blood at the beginning of the heart's relaxation phase (diastole).

As a semilunar valve, the aortic valve 100 consists in most cases of three crescent-shaped pockets. The valve lies with its bulges (sinuses) at the origin of the ascending aorta 102 (aorta ascendens). In the German language, the pockets are designated according to the outflow of the two coronary arteries from the associated sinus: right coronary pocket at the outflow of the right coronary artery, left coronary pocket at the outflow of the left coronary artery and acoronary pocket (sinus without outgoing coronary artery). With an aortic valve insufficiency and/or aortic valve endocarditis, it is often necessary to replace the native aortic valve 100 with a replacement heart valve 10.

Aortic valve endocarditis can be caused by numerous microorganisms. Particularly gram-positive bacterial species such as e.g. streptococci, enterococci and staphylococci frequently occur as human-pathogenic germs in infective endocarditis. If they colonize the endocardium, e.g. the native aortic valve 100, in the course of bacteremia, infective endocarditis develops, which is an inflammation of the inner lining of the heart (endocardium). If left untreated, the course of the disease is usually fatal.

In the context of bacterial endocarditis, tissue deposits 101 are further formed on the heart valves 100 by bacteria, their metabolites as well as other parts of the human organism. Such tissue deposits 101 are also referred to as “heart valve vegetation.” So-called non-bacterial thrombotic vegetation can be regarded as being a prerequisite for bacterial colonization. These are thrombocytes which attach to damaged endothelium of the heart valves 100.

In heart valve endocarditis, the heart valve vegetation 101 forms in particular as fibrous or membranous structures on the native heart valves 100, which can be up to 25 mm long.

The risk here is that the vegetation 101 will be torn away by the pumping heart and clog blood vessels in the organs as it flows through the bloodstream. Stroke or renal embolism are among the feared resulting complications, whereby stroke is feared the most since it carries a high risk of inflammation of the brain or cerebral membrane.

A further complication is the risk of germs being spread to other organs, where abscesses can then form. Acute organ failure can occur (for example kidney, liver and/or lung failure) as a result of blood poisoning and septic or toxic shock from toxigenic bacteria.

In order to minimize the risk posed by heart valve vegetation 101 when treating aortic valve endocarditis, the invention provides for appropriately compartmentalizing the heart valve vegetation 101 within the scope of replacing an endocarditis-diseased heart valve 100. This can preferably take place prior to replacing the native heart valve 100 with a prosthetic heart valve 10 or else during the replacement or following replacement of the diseased native heart valve 100.

An implant 1 comprising a capture device 2 is used for the compartmentalization of the heart valve vegetation 101, wherein said capture device 2 can be introduced into the patient's body in a compressed state in minimally invasive manner and expanded at the implantation site on the diseased heart valve. The capture device 2 is designed to at least partially grasp and/or capture and thus compartmentalize tissue deposits 101 (heart valve vegetation), at least in the implanted and expanded state.

In the embodiments shown in FIG. 3A to 3F, FIG. 4A to 4D and FIG. 5A to 5E, the implant 1 with the capture device 2 further comprises a corresponding replacement heart valve 10, which is either a self-expandable or balloon-expandable replacement valve able to be introduced into the patient's body in a minimally invasive manner in a compressed state and expanded at the implantation site on the diseased heart valve 100 such that the replacement heart valve 10 at least substantially assumes the function of a native heart valve 100.

The replacement heart valve 10 is preferably allocated a stent 3 in order to support and bear the replacement heart valve 10. The stent 3 allocated to the replacement valve 10 is designed to radially displace the diseased native heart valve 100 or the heart valve leaflets 103 of the diseased native heart valve 100 respectively in order for the replacement heart valve 10 to be stretched out in its place and ensure unfailing valve function during the systole and diastole of the heart.

The stent 3 allocated to the replacement valve 10 is in particular structurally designed so as to provide secure retention for the replacement heart valve 10 during the periodic beating of the heart so that the implant 1 cannot dislodge from the biological tissue, in particular from the vascular wall, and be flushed out of the implantation site due to changing pressure conditions in the heart.

The at least one stent 3 allocated to the replacement valve 10 preferably further serves as an anchoring structure for the capture device 2 of the implant 1. The use of multiple stents 3 or stent systems suitably connected or connectable to one another for these functions (carrier and supporting structure of the replacement heart valve 10 and anchoring structure of the capture device 2) is however of course also conceivable in this context.

The stent or stents 3 of the inventive implant 1 can be expanded by balloon expansion using a balloon catheter and positioned at the implantation site. The stent 3, which is compressed and encapsulated within the catheter, is thereby expanded by a catheter balloon to be filled with liquid or gas.

Alternatively, the at least one stent 3 of the inventive implant 1 can be a self-expandable stent 3. In particular, the stent 3 consists of a shape memory alloy, preferably nitinol, to that end. In addition to the shape memory effect at a specific transition temperature which is close to body temperature, nitinol also exhibits superelasticity, biocompatibility and corrosion resistance. Nitinol is thus already widely used in medical technology. Particularly the superelasticity is advantageous with respect to a stent's compressed form of delivery in the transcatheter method and the expansion at the implantation site.

In addition to the two separately executed expansion processes, a combination of both processes is likewise possible. In particular, the radial pretension of the at least one stent 3 of the implant 1 can be additionally further increased after the self-expansion by a balloon expansion, whereby a higher stability of the inventive implant 1 in the implanted state is in turn achieved.

The replacement heart valve 10 secured to the at least one stent 3 can be a pericardial valve, a biological heart valve (for example pig or cattle), an artificial heart valve 100, preferably of biocompatible materials, or a comparable implant or transplant suitable for replacing an endocarditis-diseased heart valve 100. The inventive implant 1 thus offers the advantage of the replacement heart valve 10 being able to have the optimal design depending on the patient-specific conditions.

The replacement heart valve 10 comprises at least two heart valve leaflets 103. With regard to the replacement of a three-part heart valve 100, usage with more than two, in particular three, leaflets 103 is also conceivable. The use of the inventive implant 1 is thus not limited just to the treatment and replacement of one endocarditis-diseased native aortic valve 100, particularly not by the number of leaflets 103.

In their intended use, the leaflets 103 of the replacement heart valve 10 have two positions in particular, which they assume during the systole and diastole of the heart. With the objective of mimicking a native heart valve 100 as a biological model, an equivalent conferring of the functionality of the leaflets 103 as per the biological model is accordingly also conceivable for the replacement of the other native heart valves 100. In a first position of the leaflets 103, during the diastole of the heart, the flow connection between the left ventricle and the aorta 102 is completely closed so as to prevent blood reflux. The commissures of the leaflets 103; i.e. the inner vascular edges, are thereby in contact with one another. During the systole of the heart, the leaflets 103 assume a second, opened position so that the blood can be pumped from the ventricle into the aorta 102. The commissures of the leaflets 103 have no contact with one another in this second position.

The capture device 2 of the inventive implant 1 can exhibit an anchoring structure in the form of a stent 3, wherein this stent 3 can serve as a carrier and supporting structure for the replacement heart valve 10. The capture device 2 can in this way be appropriately fixed and positioned in the implantation site.

A capturing structure 4 is connected to the anchoring structure 3 which when viewed in the direction of the blood flow, extends downstream from the anchoring structure 3 into a vessel 102 connected to the diseased heart valve 100 in terms of flow, in particular into the aorta, and exhibits at least one clamping area 5 in the expanded state which is designed to interact with at least one heart valve leaflet 103 of the diseased heart valve 100 in the expanded and implanted state of the capture device 2 such that the at least one heart valve leaflet 103 is positioned between the anchoring structure 3 and the capturing structure 4.

The clamping area 5 of the capture device 2 can have at least one clamping arm or clamping bracket designed to expand at least in part in the radial direction upon the capture device 2 being expanded in the implantation site.

According to embodiments, the clamping area 5 can exhibit at least one area 6 directed at least substantially toward the anchoring structure 3 at a distal end region facing away from the anchoring structure 3 in order to optimize a grasping or capturing of the heart valve vegetation 101 and non-positively or positively interact with an inventive structure 7 (e.g. abutment implant).

When treating or respectively replacing an endocarditis-diseased heart valve 100, in particular an aortic valve 100, a structure 7 can first be inserted prior to the introduction of the implant 1 with the capture device 2 and the replacement heart valve 10 which is of a substantially annular structure and is for example supported in the pockets of the native heart valve 100 and can fill them. This structure 7 serves as an abutment for the implant 1 to then be subsequently implanted. Preferably, the annular abutment structure 7 exhibits a longitudinal shape and height which do not block the openings of the coronary arteries in the implanted state.

The inventive implant 1 can—as depicted in the drawings—be incrementally implanted as one coherent system over the course of one insertion procedure. It is however also conceivable to implant the implant 1 and the structure 7 one after the other in a series of separate insertion procedures and in particular then; i.e. in the implanted state, connect the components of the implant 1, in particular the capture device 2 and the replacement heart valve 10 or respectively a stent allocated to the replacement heart valve 10.

In the embodiment shown in FIG. 3A to 3F, the implant is incrementally implanted over time, whereby the capture device is expanded and pushed downward and non-positively or positively connected to the abutment implant 7 in a first step and the heart valve is expanded and released in a second step.

In the embodiment shown in FIG. 4A to FIG. 4D, the implant 1 is incrementally expanded sequentially over time, whereby the replacement heart valve 10 is implanted and expanded in a first step and the capture device 2 expanded in a subsequent second step.

In contrast, the insertion procedure shown in FIG. 5A to FIG. 5E provides for the capture device 2 to be expanded first and then the replacement heart valve 10.

With both insertion procedures, the implant 1 accommodated in the compressed state in a catheter tip of an catheter introduction system 20 is advanced to the implantation site via a transfemoral approach. In principle, however, it is also conceivable for the implant 1 to be introduced via a transapical route; i.e. coming from the apex of the heart.

For implantation, preferably the annular abutment 7 is first inserted into the pockets of the native heart valve 100. However, the invention is not limited to the provision of such an abutment 7.

One embodiment of the abutment implant 7 provides for its coaxial introduction into the catheter; i.e. with radial compression and thereby axial extension over a catheter, and then its radial (self-)expansion. Another embodiment of the abutment implant 7 provides for its introduction not coaxially but rather at least substantially folded and orthogonal to the catheter axis in order for a larger volume to be introduced with the same catheter diameter.

The inventive implant 1 is thereafter implanted via a catheter 20, the catheter tip of which accommodates the implant 1 in a compressed state. Preferably, the implant 1 is accommodated in the catheter tip of the catheter introduction system 20 in a state which is stretched in the longitudinal direction of the implant 1 and reduced with respect to the radial direction of the implant 1 so as to be able to minimize the diameter of the catheter system 20.

Moreover, a guide wire 21 is preferably used in the implantation in order to make the introduction of the catheter easier and safer.

In the insertion procedure shown in FIG. 4A to 4C, the catheter tip is moved as far toward the ventricle that the replacement heart valve 10 accommodated in the catheter tip is at the height of the native heart valve 100. Then follows the expansion of a stent 3 allocated to the replacement heart valve 10 to which the replacement heart valve 10 is suitably secured.

This ensues by the catheter tip being appropriately manipulated in order to move a sleeve-like region which at least partly forms the catheter tip and holds the implant 1 in the compressed state in the proximal direction. As a result of this displacement, a part of the implant 1 is released, particularly the stent 3 allocated to the replacement heart valve 10 with the affixed replacement heart valve 10.

When the stent 3 allocated to the replacement heart valve 10 expands, it presses the native heart valve leaflets 103 of the diseased heart valve 100 in the radial direction, whereby the annular structure 7 optionally inserted previously into the pockets of the native heart valve 100 serves as an abutment.

In the state shown in FIG. 4B, it is in particular possible to check the proper positioning of the replacement heart valve 10 as well as its unfailing functionality. If it is thereby shown that the replacement heart valve 10 is not positioned in the right place, this can easily be corrected without any difficulty. On the other hand, should the replacement heart valve 10 not be functioning properly, it can be returned to the catheter sleeve and the implant 1 removed again from the patient's body.

Once the correct positioning and functioning of the replacement heart valve 10 have been verified, the capture device 2 can then be released, and that by distally displacing a corresponding second sleeve-like region of the catheter tip. The capture device 2 is preferably “programmed” such that it encloses the heart valve vegetation 101 during its expansion and thus takes it out of the main bloodstream.

In the alternative insertion procedures shown in FIG. 3A to FIG. 3F and FIG. 5A to FIG. 5E, the capture device 2 is first released from the catheter tip. The replacement heart valve 10, or the stent 3 allocated to the replacement heart valve 10 respectively, is then released.

This insertion procedure has the advantage of the heart valve vegetation 101 already being compartmentalized before the replacement heart valve 10 expands, although the insertion procedure shown in FIG. 5A to 5E necessitates the catheter tip being inserted further in the direction of the ventricle.

In the insertion procedures shown in FIG. 3A to FIG. 3F and in FIG. 5A to 5E, a clamping area 5 initially expands upon the capture device 2 being released, wherein this clamping area 5 is connected to an anchoring structure 3 of the (not yet implanted) implant 1.

Able to be seen from the depictions in FIGS. 5B and 5C is that the clamping area 5 exhibits at least one area 6 at a distal end region on the far side from the anchoring structure 3 which is substantially directed toward the anchoring structure 3, this considerably simplifying the capture and enclosure of the heart valve vegetation 101.

After the capture device 2 has been fully released and expanded (see FIG. 5D as well as FIG. 3C), the replacement heart valve 10, or a stent 3 allocated to the replacement heart valve 10 respectively, is released by manipulating a front sleeve-shaped region of the catheter tip in the proximal direction.

The implant 1 and/or the annular abutment 7 inserted into the pockets of the native heart valve 100 is/are in particular designed to release one or more active substances, in particular antibacterial, antithrombotic and cell growth-inhibiting compounds, in situ.

The invention is not limited to the embodiments depicted in the drawings but rather yields from an integrated overall consideration of all the features disclosed herein.

### LIST OF REFERENCE NUMERALS

1 implant
2 capture device
3 stent
4 capturing structure
5 clamping area
6 capturing structure area directed toward the abutment structure
7 abutment implant/abutment structure
10 prosthetic heart valve
20 catheter
21 guide wire
100 heart valve
101 heart valve vegetation/tissue deposit
102 aorta
103 (native) heart valve leaflet

1. An implant for the treatment and/or replacement of a diseased inflamed and/or infected heart valve, wherein the implant comprises a capture device which is able to be introduced into a patient's body in a compressed state in minimally invasive manner and expanded at an implantation site on the diseased heart valve, wherein the capture device, at least in an implanted and expanded state, is designed to compartmentalize tissue changes or tissue deposits in the form of heart valve vegetation or deposits by the capture device at least partially grasping and/or capturing the tissue change or deposit,
wherein the capture device comprises an anchoring structure for the fixation and/or positioning of the capture device at the implantation site, and wherein the capture device comprises a capturing structure connectable to the heart valve which extends downstream, as viewed in the direction of the blood flow, from the anchoring structure into a vessel connected to the diseased heart valve in terms of flow and exhibits at least one clamping area in the expanded state which is designed to interact with at least one heart valve leaflet of the diseased heart valve in the expanded and implanted state of the capture device such that at least one heart valve leaflet is pressed toward a vascular wall and at least partially enclosed by the capture device.

2. (canceled)

3. The implant according to claim 1,
wherein the at least one clamping area comprises at least one clamping arm or clamping bracket designed to at least partly expand radially upon the expansion of the capture device in the implantation site, wherein the clamping area designed to interact at a distal end region on the far side from the anchoring structure with a further abutment structure to be implanted.

4. The implant according to claim 1,
wherein the implant further comprises an expandable replacement heart valve which can be introduced into the patient's body in a compressed state in minimally invasive manner and is expandable at the implantation site on the diseased heart valve such that the replacement heart valve assumes the function of a native heart valve, wherein the replacement heart valve and the capture device are connected or connectable to one another by means of a common anchoring structure designed to be positioned and fixed in an area of roots of the heart valve leaflets of the native heart valve.

5. The implant according to claim 4,
wherein the implant is designed to expand incrementally over time in the implantation site on the diseased native heart valve, wherein the capture device expands in a first step and the replacement heart valve expands in a second step; or
wherein the implant is designed to expand incrementally over time in the implantation site on the diseased native heart valve, wherein the replacement heart valve expands in a first step and the capture device expands in a second step.

6. The implant according to claim 1,
wherein the implant further comprises a structure able to be implanted separately from the capture device and which exhibits a substantially annular self-expanding structure and is insertable into pockets of a native heart valve and serves as abutment for the capture device or the clamping area of the implant.

7. The implant according to, claim 1, wherein the implant and/or an abutment structure allocated to the implant is/are designed to release antimicrobial, antithrombotic or cell growth-inhibiting active substances at least in the implanted state.

8. The implant according to claim 7,
wherein for the release of the active substances, the capture device and/or a replacement heart valve that may be part of the implant and/or an abutment structure allocated to the implant has a coating of the active substances.

9. The implant according to claim 7,
wherein for the release of the active substances, the implant or components of the implant and/or the abutment structure allocated to the implant comprise at least one bioresorbable structure exhibiting a fibrous, non-woven and/or membrane structure; and/or wherein the implant or components of the implant and/or the abutment structure allocated to the implant comprise at least one active substance-releasing area which is at least partially coated or covered by at least one polymer material and designed to control the direction of release of the active substance and/or at least greatly reduce a release of the active substance toward the at least one polymer material used for the coating.

10. A catheter for introducing the implant of claim 1 into a body of a patient, wherein the catheter has a catheter tip able to be manipulated via a handle of the catheter such that the implant can be released from the catheter tip incrementally.

11. A system for the treatment or the replacement of an inflamed, thrombosed or degenerated heart valve comprising the implant according to claim 1 and a catheter, wherein the catheter has a catheter tip able to be manipulated via a handle of the catheter such that the implant can be released from the catheter tip incrementally, and wherein the implant is designed to be able to be accommodated in the catheter tip when the implant is extended in the longitudinal direction and in a reduced state relative to the radial direction of the implant, and wherein the catheter tip is designed to accommodate the implant when the implant is extended in the longitudinal direction and in the reduced state relative to the radial direction of the implant.

12. The system according to claim 11,
wherein the system comprises an additional and/or independent at least substantially annular, self-expanding implant which is designed to serve as an abutment for the implant of claim 1 and which is able to be folded by radial stretching and accommodated in folded form in the catheter and implanted at the implantation site such that in the implanted state of the implant, at least some areas of at least one valve leaflet of the heart valve to be treated can be accommodated between the implant of claim 1 and the additional and/or independent implant.

13. The system according to claim 12,
wherein the additional and/or independent implant is designed to be receivable in the catheter tip in the compressed state such that the additional and/or independent implant in the compressed state is not aligned coaxially but rather orthogonally to the catheter tip axis and is also able to be incrementally released at the implantation site by means of one or more pressure-resistant connections.

14. A method for the treatment or the replacement of an inflamed, thrombosed, or degenerated heart valve, wherein the method comprises the method step of compartmentalizing tissue changes or tissue deposits in the form of heart valve vegetation or deposits, and by the tissue changes or tissue deposits being at least partially captured and/or grasped by a capture device.

15. The method according to claim 14,
wherein the method further comprises the method step of implanting a replacement heart valve to replace the diseased native heart valve, wherein the step of compartmentalizing and the step of implanting the replacement heart valve are preferably performed chronologically sequentially; and/or
wherein the method further comprises the method step of releasing antimicrobial, antithrombotic and cell growth-inhibiting active substances, wherein the release ensues in situ.
